(19) United States
(12) Patent Application Publication
(10) Pub. No.: US 2013/0142745 A1
(43) Pub. Date: Jun. 6, 2013

(54) COSMETIC COMPOSITION FOR LIP MAKE-UP

(71) Applicants: Laurence Jamin, Rueil Malmaison (FR); Helene Villecroze, Sannois (FR)

(72) Inventors: Laurence Jamin, Rueil Malmaison (FR); Helene Villecroze, Sannois (FR)

(73) Assignee: CHANEL PARFUMS BEAUTE, Neuilly sur Seine (FR)

(21) Appl. No.: 13/816,024

(22) PCT Filed: Sep. 8, 2011
(86) PCT No.: PCT/EP2011/065580

§ 371 Date: Feb. 8, 2013

(30) Foreign Application Priority Data
Sep. 13, 2010 (FR) 10 57269

(51) Int. Cl. A61K 8/92

(52) U.S. Cl. CPC A61K 8/92
USPC 424/64

(57) ABSTRACT

A cosmetic composition particularly intended to be cast into a cup with view to a use as a lip gloss, contains: (a) at least one glossy oil; (b) at least one ester obtained from: (i) a saturated or unsaturated, linear or branched aliphatic acid or hydroxy acid, having from 8 to 30 carbon atoms, (ii) a linear or branched diacid having from 12 to 36 carbon atoms, and (iii) glycerol or a glycerol condensate; (c) at least one hydrophobicized pyrogenated silica; (d) at least one wax consisting of a homo- or co-polymer of ethylene; and (e) optionally stearic acid. The lip gloss, and a lip care or make-up cosmetic method including the topical application of this composition on the lips are also described.

The present invention relates to a cosmetic composition in particular intended to be cast into a container, such as a cup, with view to a use as a lip gloss. It also relates to a cosmetic method for taking care of lips or making up lips, comprising topical application on the lips of this composition.

Among lip make-up compositions, lip glosses or “gloss” designate transparent to opalescent compositions intended to provide gloss to the lips by the use of certain oils having a high refractive index. These compositions are generally conditioned in a container such as a small flask provided with an applicator, generally a foam pencil. Alternatively, they may be cast into a cup, in particular when these compositions are intended to be incorporated in a make-up palette.

These products for lips should all allow deposition on the lips of a glossy, flexible film neither too fat, nor too dry, not very tacky, and having satisfactory hold. In addition that achieving these goals is a first challenge to take up, the formulation of lip glosses in a cast form moreover implies that a certain number of additional constraints be taken into account, related to how these products are presented and used. Thus, they should have a sufficiently soft texture so that their taking up with the pencil is easy, while having sufficient consistency so as not to run when the cups containing them are in a vertical position in a handbag or in a suitcase, which is particularly critical in the case of makeup palettes, which are designed for travel. Further, in order to preserve the attractiveness of the product, the latter should have a sufficiently smooth and glossy aspect in its container and the oils which it contains should not exude.

It appeared that formulating a lip gloss meeting all these constraints was a real challenge. In particular, if the texture of these products may be adjusted and their exudation limited by adding waxes and/or oily gelling agents, the latter however tend to matify the composition, which of course is not desirable.

After many investigations, the Applicant however managed to develop a lip gloss formula which has a glossy aspect, a consistency adapted to use in the cast form and absence of exudation.

The object of the present invention is therefore a cosmetic composition comprising, in a physiologically acceptable medium:

(a) at least one glossy oil;

(b) at least one ester obtained from: (i) an aliphatic, saturated or unsaturated, linear or branched acid or hydroxy acid having from 8 to 30 carbon atoms, (ii) a linear or branched diacid having from 12 to 36 carbon atoms, and (iii) glycerol or a glycerol condensate;

(c) at least one hydrophobicized pyrogenated silica;

(d) at least one wax consisting of a homo- or copolymer of ethylene;

(e) optionally stearic acid.

One skilled in the art will be able to adjust the amounts of the various ingredients above depending on the nature of the selected oils, as well as on the level and on the nature of the powdery materials to be incorporated in order to be able to obtain the sought hue.

These ingredients will now be described in more detail.

### Oils

In the sense of the present invention, by <<oil>> is meant a compound which is liquid at room temperature (25° C.), and which, when it is introduced in an amount of at least 1% by weight in water at 25° C., is soluble in an amount of less than 10% by weight, preferably less than 5% by weight and better not at all soluble in water, based on the weight of oil introduced into the water.

Further, by <<glossy oil>>, is meant an oil having a refractive index greater than 1.45 and preferably greater than 1.47.

Examples of glossy oils are notably hydrocarbon oils such as polybutene, hydrogenated polyisobutene or hydrogenated polydecene, as well as phenylated silicone oils, such as those identified by the INCI name of <<phenyl trimethicone>>, an example of which is formed by the silicone available under the trade name of MIRASIL® PTM from RHODIA, those identified by the INCI name of <<phenylpropyldimethylsiloxysilicate>>, an example of which is formed by the silicone available under the trade name of SILSHINE® 151 from GENERAL ELECTRIC and those identified by the INCI name of <<trimethyl pentaphenyl trisiloxane>>, an example of which is formed by the silicone available under the trade name of DC Pe 1555 HRI from DOW CORNING.

As glossy oils, mention may also be made of fluorinated silicones such as those identified by the INCI name of perfluorononyl dimethicone, an example of which is formed by the silicone available under the trade name of PECOSIL® FS (FSU, FSL . . . ) from PHOENIX and another example is formed by the silicone available under the trade name of Biosil Basics® (Fluorosil LF, 14 . . . ) from BIOSIL TECHNOLOGIES.

Other examples of glossy oils are vegetable oils and in particular castor seed oil; branched and/or unsaturated fatty alcohols such as octyldodecanol; mono- and poly-esters of fatty acids and/or of fatty alcohols, the fatty chain of which contains from 6 to 20 carbon atoms, and in particular: mono- and poly-esters of hydroxy acids and fatty alcohols such as diisostearyl malate, benzoic acid esters of fatty alcohols such as C₁₂-C₁₅ alkyl benzoates, polyolpolyesters and notably (di)pentaerythrityl polyesters such as pentaerythrityl tetraisostearate, dipentaerythrityl pentaisononanoate and C₅-C₉ dipentaerythrityl or polyglycerol esters, such as the one known under the INCI name of <<bis-diglyceryl polyacyladipate-1>>, marketed by SASOL under the trade name of SOFTISAN® 645, or trimethylolpropane, such as trimethylopropane triethylhexanoate which is notably marketed by KOKYU ALCOHOL KOGYO under the trade name of KAK® TTO, or propylene glycol esters, such as propylene glycol dibenzoate which is notably marketed by INOLEX under the trade name of LEXFEEL SHINE®, and isocetyl stearoyl stearate; and polyesters of hydrogenated castor oil such as the esters marketed by KOKYU ALCOHOL KOGYO under the trade names of RISOCAST® DA-H and RISOCAST® DA-L.

It is quite obvious that the composition according to the invention may comprise a mixture of several of the oils mentioned above.

The composition according to the invention may advantageously comprise from 15 to 50% by weight, preferably from 20 to 40% by weight of a glossy oil, based on the total weight of the composition.

### Glyceryl Ester

The composition according to the invention contains as a second essential constituent, at least one ester obtained from: (i) a saturated or unsaturated, linear or branched, aliphatic acid or hydroxy acid, having from 8 to 30 and preferably from 12 to 22 carbon atoms, (ii) a linear or branched diacid having from 12 to 36 and preferably from 12 to 20 carbon atoms, and (iii) glycerol or a glycerol condensate.

The aliphatic acid (i) may notably be selected from lauric, myristic, palmitic, oleic, stearic, 12-hydroxystearic, behenic acids and mixtures thereof. Further, the diacid (ii) may for example be selected from eicosadioic acid, ethyloctadecane-dioic acids, dodecane-dioic acids and mixtures thereof.

A glyceryl ester of this type, preferred for a use in the present invention, is the diester of eicodadioic acid and of glycerol esterified by behenic acid. This compound is an ester of formula RO—CH₂—CH(OR)—CH₂—O—CO—(CH₂)₁₈—CO—O—CH₂—CH(OR)—CH₂—OR, wherein the groups R independently designate a group —CO—(CH₂)₂₀—CH₃ or H, with the proviso that at least one of the groups R is distinct from H, or a mixture of such esters. In particular it is available from NISSHIN OILLIO under the trade name of NOMCORT® HK-G.

The ester (b) may represent from 0.5% to 10% by weight, preferably from 2 to 6% by weight, based on the total weight of the composition.

### Silica

The composition according to the invention, contains as constituent (c) at least one hydrophobicized pyrogenated silica.

By “pyrogenated silica” also designated by “fumed silica” or “sublimated silica”, is meant a silica made by hydrolysis of chlorosilanes in an oxyhydric flame. The latter is treated so as to be made hydrophobic, advantageously with dimethyldichlorosilane or with hexamethyldisiloxane, and preferably with dimethyl-dichlorosilane. The obtained hydrophobicized silica preferably has an average particle size from 5 to 20 nm, more preferentially from 10 to 20 nm, and/or a specific surface area from 50 to 300 m²/g, more preferentially from 80 to 140 m²/g.

Examples of hydrophobicized pyrogenated silicas are those designated under the INCI names of SILICA SILYLATE and SILICA DIMETHYL SILYLATE. Silicas of this type are notably marketed by DEGUSSA under the trade names of AEROSIL® R812 and AEROSIL® R 972, respectively.

The silica (c) may represent from 2 to 7% by weight and more preferentially from 3.5 to 5% by weight based on the total weight of the composition.

The Applicant observed that the presence in the composition according to the invention of hydrophobicized pyrogenated silica allowed reduction or even suppression of exudation at the surface of the composition.

### Polyethylene Wax

The composition according to the invention further contains as constituent (d), an ethylene homo- or copolymer wax, designated hereafter by the term “polyethylene wax”.

By <<wax>> is meant a fat with a reversible liquid/solid state change, having a melting temperature above 30° C. and generally less than 90° C., which is liquid under the conditions for preparing the composition and has an anisotropic crystalline organization in the solid state.

The polyethylene wax used in the composition according to the invention may be an ethylene homopolymer, advantageously having a melting point from 75 to 85° C., such as the one marketed by HONEYWELL under the trade name of ASENSA® PR 200 or further the one marketed by NEW PHASE under the trade name of Performalene® 500. Alternatively, this may be an ethylene copolymer such as an ethylene/vinyl acetate copolymer, for example the one marketed by HONEYWELL under the trade name of ASENSA® PR 220.

The polyethylene wax used in the present invention may represent from 1% to 6% by weight, and preferably from 1 to 2% by weight, based on the total weight of the composition.

This composition further contains, as an optional constituent (e), stearic acid. For purposes of simplicity, it may optionally be possible to use stearic acid premixed with a polyethylene wax. A raw material of this type is available from HONEYWELL under the trade name of ASENSA® PR 210.

The constituents described above are conveyed in the fatty phase of the composition according to the invention, which may be an anhydrous composition or an emulsion, in particular a water-in-oil (W/O) emulsion. It is preferred that the composition according to the invention be anhydrous.

In addition to these constituents, the fatty phase may contain at least one non-glossy oil among which mention may be made of: synthetic (poly)esters and (poly)ethers and in particular (poly)esters of C₆-C₂₀ acids and of C₆-C₂₀ alcohols, advantageously branched, such as isononyl isononanoate; branched and/or unsaturated fatty acids; silicone oils such as linear polydimethylsiloxanes; as well as mixtures thereof.

The fatty phase of the composition according to the invention may also contain one or more slurry compounds, i.e. lipophilic fats which, like waxes, are capable of undergoing a reversible liquid/solid state change and have an anisotropic crystalline organization in the solid state, but which are different from waxes by the fact that they contain, at a temperature of 23° C., a liquid fraction and a slurry fraction. A compound of this type is notably a mixture of esters of sterols, such as the mixture of cholesterol and lanosterol esters available from CRODA under the trade name of Super Sterol Ester®.

The composition according to the invention may further contain at least one filler. By this term is meant any particle of any shape (notably spherical or lamellar), either mineral or organic, insoluble in the composition. Examples of fillers are talc, mica, silica, kaolin, boron nitrite, starch, starch modified by octenylsuccinic anhydride, polyamides, silicone resins, silicone elastomer powders and acrylic polymer powders, in particular of poly(methyl methacrylate). The fillers may notably consist of several layers of a different chemical nature and/or of a different physical shape and notably appear as lamellas coated with spherical fillers. They may be modified by means of different surface treatments. An exemplary filler treated at the surface is formed by silica modified by an ethylene/methacrylate copolymer notably marketed by KOBO under the trade names of DSPCS® 20N-I2, 3H-I2 and I2.

The composition may further contain at least one coloring material selected from water-soluble or oil-soluble coloring agents, the fillers having the effect of coloring and/or opacifying the composition and/or coloring the lips, such as pigments, mother-of-pearls, lacquers (water-soluble coloring agents adsorbed on an inert mineral support) and mixtures thereof. These coloring materials may optionally be treated at the surface by a hydrophobic agent such as silanes, silicones, fatty acid soaps, C₉-C₁₅ fluoroalcohol phosphates, acrylate/dimethicone copolymers, mixed C₉-C₁₅ fluoroalcohol phosphates/silicone copolymers, lecithins, carnauba wax, polyethylene, chitosan and amino acids optionally acylated such as lauroyl lysine, disodium stearoyl glutamate and aluminium acyl glutamate. The pigments may either be mineral or organic, natural or synthetic. Examples of pigments are notably iron, titanium or zinc oxides, as well as composite pigments and goniochromatic, pearlescent, interferential, photochromic or thermochromic pigments, without this list being a limitation. Examples of pigments which may be used in the composition according to the invention are hemispherical composite pigments made from cross-linked poly(methylacrylic acid) methyl ester and from organic coloring agents. Such composite pigments are notably marketed by DAITO KASEI. The mothers-of-pearls may be selected from those conventionally present in makeup products, such as mica/titanium dioxide products. Alternatively, these may be mothers-of-pearls based on mica/silica/titanium dioxide, based on synthetic fluorphlogopite/titanium dioxide (SUNSHINE® from MAPRECOS), calcium sodium borosilicate/titanium dioxide (REFLECKS® from ENGELHARD) and calcium aluminium borosilicate/silica/titanium dioxide (RONASTAR® from MERCK).

The Applicant observed that the presence of mica in the composition according to the invention allows reduction in exudation.

The Applicant further observed that the presence in the composition according to the invention of mica provided with an organic coating allowed reduction or even suppression of the occurrence of cracks at the surface of the composition, which may appear in particular when the latter does not contain any other mother-of-pearl. Thus, according to a preferred embodiment, the composition further contains at least one filler based on mica coated with an organic salt, in particular magnesium myristate. Fillers of this type are notably available commercially from KOBO PRODUCTS under the trade name of MICA S/MM³®, or from SENSIENT under the trade name of SERECITE HLC®. This filler based on coated mica may represent from 3 to 8% by weight, and preferably from 4 to 6% by weight, based on the total weight of the composition.

Advantageously, when it contains one or more pigments, the composition according to the invention further contains at least one dispersant such as butylene glycol cocoate or diisostearyl malate.

The composition according to the invention may also contain one or more sweeteners such as sorbitol, saccharose, xylitol, acesulfam K and sodium saccharinate; antioxidants such as ascorbic acid and/or its alkylated or phosphorylated esters and/or tocopherol and/or its esters; sequestering agents such as EDTA salts; pH adjusters; preservatives; and perfumes.

Examples of such adjuvants are notably mentioned in the CTFA Dictionary (International Cosmetic Ingredient Dictionary and Handbook published by The Cosmetic, Toiletry and Fragrance Association, Second Edition, 2006).

Preferably, the composition according to the invention is used as a lip make-up and/or care product, for example as a <<gloss>> or lip gloss.

The object of the present invention is therefore also a lip care and/or make-up cosmetic method, comprising the topical application on the lips of the composition as described earlier.

The object thereof is also a lip gloss in the form of a container, such as a cup, in which a cosmetic composition is cast such as the one described earlier.

The invention will now be illustrated by the following non-limiting examples.

### EXAMPLES

### Example 1

### Lip Gloss

In a standard way for one skilled in the art, a lip gloss was made having the following composition in which the proportions of the ingredients are expressed in weight percentages:

| Ingredient | |
|---|---|
| INCI name/type | Amount |
| C10-30 CHOLESTEROL/LANOSTEROL ESTERS | 36% |
| OCTYLDODECANOL | 9% |
| DIISOSTEARYL MALATE | 6.4% |
| SILICA DIMETHYL SILYLATE | 2.5% |
| POLYBUTENE | 30% |
| POLYETHYLENE & STEARIC ACID (50/50) | 3% |
| GLYCERYL BEHENATE/EICOSADIOATE | 4% |
| Preservatives | |
| Pigments (milled in diiostearyl malate) | 5% |
| and mothers-of-pearls | |
| MICA & MAGNESIUM MYRISTATE | 3% |
| Antioxidants | 0.1% |

This lip gloss was cast in a cup integrated to a make-up palette.

### Example 2

### Comparative Study

### Influence of the Wax

Different lip glosses were formulated, having the composition indicated in Table 1 below, while varying the nature of the waxes.

For each of these formulations, the aspect of the product in the pot (glossiness and exudation) and the sensorial characteristics of the product were evaluated upon application.

The glossiness and the exudation were evaluated visually. For measuring exudation, the number and the size of the drops formed on the product were specifically evaluated after the latter had been kept for 15 days at 20° C. The terms of observations are 1 h, 2 h, 4 h, and 24 h at different temperatures (27° C., 30° C., 35° C. and 45° C.). A sample is considered as not exuding if it has at least 2 to 3 drops of a small size at 27° C. and 30° C. and preferably under all these conditions.

The results of these tests are also grouped in Table 1.

TABLE 1

| Ingredient: | | | | |
|---|---|---|---|---|
| chemical name/INCI name | No.1 | No.2 | No.3 | No.4 |
| Synthetic wax | 6% | — | — | — |
| (Sasol ® Wax C80), | | | | |
| MP > 88° C. | | | | |
| Hydrogenated Castor oil | — | 6% | — | — |
| (Castorwax MP 80), | | | | |
| MP = 77-83° C. | | | | |
| Microcrystalline | — | — | 6% | — |
| wax | | | | |
| (Cerewax BL A75), | | | | |
| MP = 63-73° C. | | | | |
| Polyethylene wax | — | — | — | 6% |
| (Asensa PR200) | | | | |
| C10-30 | 20.9% | 20.9% | 20.9% | 20.9% |
| CHOLESTEROL/ | | | | |
| LANOSTEROL ESTERS | | | | |
| OCTYLDODECANOL | 9.1% | 9.1% | 9.1% | 9.1% |
| DIISOSTEARYL | 4.45% | 4.45% | 4.45% | 4.45% |
| MALATE | | | | |
| SILICA DIMETHYL | 2.45% | 2.45% | 2.45% | 2.45% |
| SILYLATE | | | | |
| POLYBUTENE | 49.9% | 49.9% | 49.9% | 49.9% |
| Antioxidants | 0.1% | 0.1% | 0.1% | 0.1% |
| Preservative | 0.1% | 0.1% | 0.1% | 0.1% |
| 10% milled | 7% | 7% | 7% | 7% |
| pigments in the | | | | |
| remainder of the | | | | |
| composition | | | | |
| Observations | Does not | Glossy, | Mat | Glossy |
| | exude, | easy to | product | product, |
| | good | apply, but | which | good grip, |
| | adherence | tacky and | exudes | does not |
| | but tacky | too soft | | exude, soft |
| | mat and | product | | texture |
| | texture | which | | |
| | | exudes | | |

It emerges from this table that the polyethylene wax provides better results, in terms of glossiness and exudation than the other waxes, even those having a close melting point and/or a same synthetic origin. On the other hand, the texture of the product remains too soft. A portion of the polyethylene wax was then substituted with a fatty phase gelling agent, Nomcort® HK-G. An improvement in the consistency of the product is observed, which has a homogeneous texture and suitable hardness, without negatively effecting glossiness. On the other hand, the association of this gelling agent with other waxes did not give the possibility of obtaining the sought compromise of properties.

### Example 3

### Comparative Example

### Vertical Test

The compositions described in Table 2 below were prepared.

TABLE 2

| Ingredient: | | | |
|---|---|---|---|
| chemical name/INCI name | A | B | C |
| C10-30 CHOLESTEROL/ | Qsp | Qsp | qsp |
| LANOSTEROL ESTERS | | | |
| OCTYLDODECANOL | 9% | 9% | 9% |
| DIISOSTEARYL MALATE | 6.4% | 6.4% | 6.4% |
| SILICA DIMETHYL | 2.5% | 2.5% | 3.5% |
| SILYLATE | | | |
| POLYBUTENE | 30% | 30% | 30% |
| POLYETHYLENE | 3% | 1.5% | 1.5% |
| STEARIC ACID | — | 1.5% | 1.5% |
| GLYCERYL BEHENATE/ | 4% | 4% | 4% |
| EICOSADIOATE | | | |
| Antioxidants | 0.1% | 0.1% | 0.1% |
| 20% to 40% milled | 0-6.5% | 0-6.5% | 0-6.5% |
| pigments in the oil | | | |
| MICA & MAGNESIUM | 3% | 3% | 3% |
| MYRISTATE | | | |
| Vertical Test: | | | |
| time | 1 h/4 h/24 h | 1 h/4 h/24 h | 1 h/4 h/24 h |
| observations | 0 drop/a | 0 drop/0 | 0 drop/0 |
| | few drops/ | drop/2 | drop/0 |
| | 4-5 drops | drops | drop |

The comparison of formulations A and B show that by substituting a portion of the polyethylene wax with stearic acid, it is possible to improve the stability of the product in the vertical position at 45° C. over 24 hours. An exudation test was carried out under the conditions described in Example 2 on the composition B and showed that stearic acid gives better results in terms of exudation at 35° C. and 45° C., relatively to the composition A. As this emerges from the comparisons of formulations B and C, the latter may further be improved by adjusting the hydrophobic silica level, until no exudation drop is either observable on the surface or on the edges of the cup.

Moreover the glossiness of formula B was evaluated by means of a Samba® apparatus marketed by BOSSA NOVA TECHNOLOGIES (Venice, USA), by using the following adjustments: application to the pneumatic applicator; deposit of 250 μm; drying time of 4 h at 20° C.; application on contrast maps (black and white) from BYK (ref. 2810). The greater the reflectance peak (specular light), more the product is glossy. The reflectance at 0° measured for formula B was 339, characteristics of a highly glossy product.

1. A cosmetic composition comprising in a physiological acceptable medium:
(a) at least one glossy oil;
(b) at least one ester obtained from: (i) a saturated or unsaturated, linear or branched, aliphatic acid or hydroxy acid having from 8 to 30 carbon atoms, (ii) a linear or branched diacid having from 12 to 36 carbon atoms, and (iii) glycerol or a glycerol condensate;
(c) at least one hydrophobicized pyrogenated silica;
(d) at least one wax consisting of a homo- or co-polymer of ethylene;
(e) optionally stearic acid.

2. The composition according to claim 1, characterized in that the oil is selected from: hydrocarbon oils, phenylated silicone oils, fluorinated silicones, vegetable oils, mono- and poly-esters of fatty acids and/or fatty alcohols, the fatty chain of which contains from 6 to 20 carbon atoms, polyesters of hydrogenated castor oil, and mixtures thereof.

3. The composition according to claim 1, characterized in that the aliphatic acid (i) is selected from lauric, myristic, palmitic, oleic, stearic, 12-hydroxysteric, behenic acids and mixtures thereof.

4. The composition according to claim 1, characterized in that the diacid (ii) is selected from eicosadioic, ethyloctadecane-dioic, dodecane-dioïc acids and mixtures thereof.

5. The composition according to claim 1, characterized in that the ester (b) is a diester of eicosadioic acid and of glycerol esterified by behenic acid.

6. The composition according to claim 1, characterized in that the silica is treated with dimethyldichlorosilane or by hexamethyldisiloxane, preferably by dimethyldichlorosilane.

7. The composition according to claim 1, characterized in that said wax (d) is a homopolymer of ethylene having a melting point comprised between 75 and 85° C.

8. The composition according to claim 1, characterized in that it further contains at least one filler based on mica, coated with an organic salt.

9. The composition according to claim 1, characterized in that it is anhydrous.

10. The composition according to claim 1, characterized in that it is casted in a container, such as a cup.

11. A lip gloss cast in a container, such as a cup, comprising the cosmetic composition according to claim 1.

12. A cosmetic product for lip care and/or make-up, comprising the topical application on the lips of the composition according to claim 1.

13. Method of taking care of lips or making up lips, which comprises topically applying on the lips an effective amount of a composition according to claim 1.
